(19) United States
(12) Patent Application Publication
(10) Pub. No.: US 2025/0308649 A1
(43) Pub. Date: Oct. 2, 2025

(54) METHODS AND SYSTEMS FOR OUT-OF-DISTRIBUTION DETECTION IN HISTOPATHOLOGY MEDIA

(71) Applicant: AIRAMATRIX PRIVATE LIMITED, Thane (W) (IN)

(72) Inventors: Nitin SINGHAL, Thane (IN); Geetank RAIPURIA, Thane (IN); Raghav Praneeth AMARAVADI, Thane (IN); Malini ROY, Thane (IN)

(73) Assignee: AIRAMATRIX PRIVATE LIMITED, Thane (W) (IN)

(21) Appl. No.: 19/097,058

(22) Filed: Apr. 1, 2025

(30) Foreign Application Priority Data
Apr. 1, 2024 (IN) 202421027118

(51) Int. Cl. G16H 10/40; G06T 7/00

(52) U.S. Cl. CPC G16H 10/40; G06T 7/0014; G06T 2207/20081

(57) ABSTRACT

Embodiments herein disclose methods and ystems for performing unsupervised out-of-distribution detection of abnormal regions in histopathology media using multi-class in-distribution modelling. Embodiments herein disclose methods and systems for automatically identifying abnormal regions in a tissue whole slide media by utilizing a multi-class normal representation that is learned exclusively from normal tissue whole slide media.

### CROSS REFERENCE TO RELATED APPLICATION

This application is based on and derives the benefit of Indian Provisional Application IN202421027118, the contents of which are incorporated herein by reference.

### TECHNICAL FIELD

Embodiments disclosed herein relate to histopathology media, and more particularly to unsupervised detection of anomalies in histopathology media.

### BACKGROUND

Traditional supervised learning techniques rely on labeled datasets, where each data sample is explicitly categorized as either normal or abnormal. These methods operate by learning decision boundaries that distinguish between the two classes based on the provided training data. However, supervised approaches face significant limitations when applied to anomaly detection.

One of the primary challenges lies in the nature of abnormal data. Unlike normal data, which typically follows a well-defined distribution, abnormalities can arise in countless, often unpredictable ways. This makes it practically impossible to generate a comprehensive dataset that includes labeled examples for every possible abnormal scenario. As a result, supervised models trained on a limited set of abnormal cases may fail to generalize effectively to novel or previously unseen anomalies. Additionally, acquiring and labeling abnormal data can be costly, time-consuming, and sometimes infeasible, especially in domains, where anomalies are rare and diverse (for example, healthcare, manufacturing, cybersecurity, and so on).

These constraints make supervised techniques less suitable for anomaly detection, leading to the exploration of alternative approaches, such as unsupervised and semi-supervised learning, which can identify deviations from normal patterns without requiring exhaustive labeled datasets.

In the context of tumour identification in oncology, for example, there exist rare tumour types such as urothelial carcinoma and neuro-endocrine tumours in prostate cancer. These rare tumour types pose a considerable challenge to supervised algorithms due to the difficulty in obtaining samples for them. Furthermore, the definition of a non-malignant tumour is broad, encompassing benign tumours, atrophy, Atypical Small Acinar Proliferation (ASAP), and other related conditions.

Conventional supervised learning models can be trained to detect abnormalities in Whole Slide Images (WSIs). These models have a high level of memory for known anomalies. Nevertheless, the process of training a supervised model necessitates a substantial dataset that is annotated on a big scale for each specific anomaly type. This creates a barrier in constructing a computational model, as it requires inputs from an expert pathologist to analyze many WSIs. In addition, the model may come across unfamiliar tissue representations, making it impractical to create a labelled dataset for every potential anomalous scenario. Therefore, it is crucial to devise an unsupervised method that does not rely on labelled data for every conceivable normal and abnormal tissue types.

Anomaly detection has been explored using a One-Class Classifier (OCC), which aims to differentiate normal tissue from abnormal tissue by treating all normal tissue samples as a single, unified category. This approach assumes that normal tissue has a well-defined distribution, enabling the model to identify deviations as anomalies. However, in histopathology, normal tissue can exhibit diverse sub-morphologies depending on the organ, making a single-class representation overly simplistic.

One of the key limitations of this approach is its inability to capture the complexity and heterogeneity of normal tissue structures. For instance, in a given organ, multiple distinct tissue types may exist, each with unique morphological characteristics. By collapsing all normal variations into a single class, the model may fail to represent the full spectrum of normal tissue structures accurately. This can lead to misclassifications, particularly when encountering rare or less dominant tissue types that were underrepresented during training.

Furthermore, a major challenge arises from the dominance of a primary tissue type within an organ. For example, in liver tissue, the parenchyma is the predominant structure, while other components, such as bile ducts or blood vessels, occupy a much smaller portion of the sample. A one-class classifier, heavily influenced by the dominant tissue type, may struggle to learn meaningful representations of these less common but still normal structures. As a result, the model may mistakenly classify them as anomalies or fail to detect subtle abnormalities embedded within these less-represented regions.

Additionally, the unconstrained nature of the in-distribution space in OCC can lead to high misclassification rates. Since the model does not explicitly learn an abnormal class, it may struggle to define precise decision boundaries, increasing the likelihood of false positives and false negatives. FIG. 1 illustrates this issue, showing how a one-class classifier can lead to poor separation between normal and abnormal regions due to the broad and unrestricted in-distribution space. These limitations highlight the need for more sophisticated anomaly detection approaches that can better capture the diversity of normal tissue while effectively identifying pathological deviations.

Hence, there is a need in the art for solutions which will overcome the above mentioned drawback(s), among others.

### OBJECTS

The principal object of embodiments herein is to disclose methods and systems for performing unsupervised out-of-distribution detection of abnormal regions in histopathology media using multi-class in-distribution modelling.

Another object of embodiments herein is to disclose methods and systems for automatically identifying abnormal regions in a tissue whole slide media by utilizing a multi-class ponormal representation that is learned exclusively from normal tissue whole slide media.

These and other aspects of the embodiments herein will be better appreciated and understood when considered in conjunction with the following description and the accompanying drawings. It should be understood, however, that the following descriptions, while indicating at least one embodiment and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF FIGURES

Embodiments herein are illustrated in the accompanying drawings, throughout which like reference letters indicate corresponding parts in the various figures. The embodiments herein will be better understood from the following description with reference to the following illustratory drawings. Embodiments herein are illustrated by way of examples in the accompanying drawings, and in which:

FIG. 1 depicts an example one class distribution, according to existing arts;

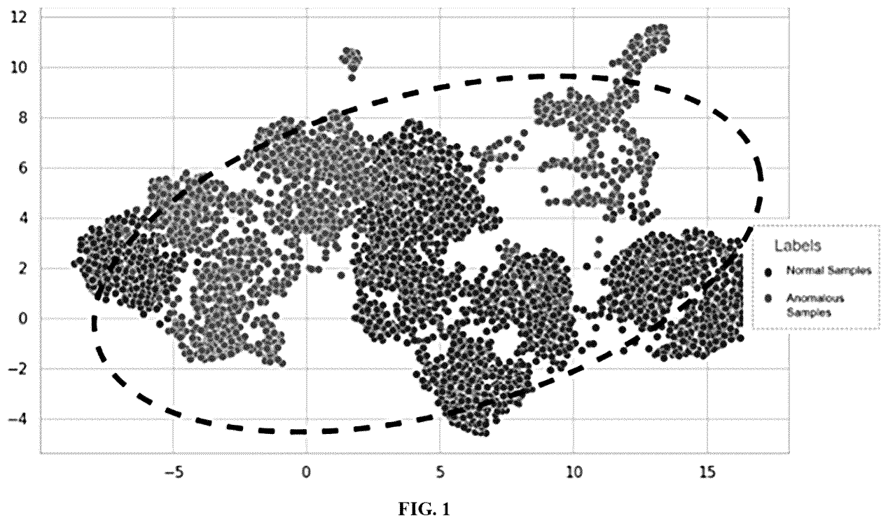

FIG. 2 depicts an example process for performing unsupervised out-of-distribution detection of abnormal regions in histopathology media using multi-class in-distribution modelling, according to embodiments as disclosed herein;

FIG. 3 depicts a system for performing unsupervised out-of-distribution detection of abnormal regions in a tissue sample, according to embodiments as disclosed herein;

FIG. 4 is a flowchart depicting the process of training a classification model, according to embodiments as disclosed herein;

FIG. 5 is a flowchart depicting the process of detecting at least one anomaly/abnormality in a tissue sample, according to embodiments as disclosed herein;

FIG. 6A presents a two dimensional (2D) representation of a multi-class normal distribution alongside abnormal class samples, visually distinguishing normal variations from anomalies, according to embodiments as disclosed herein; and

FIG. 6B overlays clustered abnormal class samples specifically from kidney tissue, illustrating how the unsupervised model detects and groups abnormalities, according to embodiments as disclosed herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

For the purposes of interpreting this specification, the definitions (as defined herein) will apply and whenever appropriate the terms used in singular will also include the plural and vice versa. It is to be understood that the terminology used herein is for the purposes of describing particular embodiments only and is not intended to be limiting. The terms “comprising”, “having” and “including” are to be construed as open-ended terms unless otherwise noted.

The words/phrases “exemplary”, “example”, “illustration”, “in an instance”, “and the like”, “and so on”, “etc.”, “etcetera”, “e.g.,”, “i.e.,” are merely used herein to mean “serving as an example, instance, or illustration.” Any embodiment or implementation of the present subject matter described herein using the words/phrases “exemplary”, “example”, “illustration”, “in an instance”, “and the like”, “and so on”, “etc.”, “etcetera”, “e.g.,”, “i.e.,” is not necessarily to be construed as preferred or advantageous over other embodiments.

Embodiments herein may be described and illustrated in terms of blocks which carry out a described function or functions. These blocks, which may be referred to herein as managers, units, modules, hardware components or the like, are physically implemented by analog and/or digital circuits such as logic gates, integrated circuits, microprocessors, microcontrollers, memory circuits, passive electronic components, active electronic components, optical components, hardwired circuits and the like, and may optionally be driven by a firmware. The circuits may, for example, be embodied in one or more semiconductor chips, or on substrate supports such as printed circuit boards and the like. The circuits constituting a block may be implemented by dedicated hardware, or by a processor (e.g., one or more programmed microprocessors and associated circuitry), or by a combination of dedicated hardware to perform some functions of the block and a processor to perform other functions of the block. Each block of the embodiments may be physically separated into two or more interacting and discrete blocks without departing from the scope of the disclosure. Likewise, the blocks of the embodiments may be physically combined into more complex blocks without departing from the scope of the disclosure.

It should be noted that elements in the drawings are illustrated for the purposes of this description and ease of understanding and may not have necessarily been drawn to scale. For example, the flowcharts/sequence diagrams illustrate the method in terms of the steps required for understanding of aspects of the embodiments as disclosed herein. Furthermore, in terms of the construction of the device, one or more components of the device may have been represented in the drawings by conventional symbols, and the drawings may show only those specific details that are pertinent to understanding the present embodiments so as not to obscure the drawings with details that will be readily apparent to those of ordinary skill in the art having the benefit of the description herein. Furthermore, in terms of the system, one or more components/modules which comprise the system may have been represented in the drawings by conventional symbols, and the drawings may show only those specific details that are pertinent to understanding the present embodiments so as not to obscure the drawings with details that will be readily apparent to those of ordinary skill in the art having the benefit of the description herein.

The accompanying drawings are used to help casily understand various technical features and it should be understood that the embodiments presented herein are not limited by the accompanying drawings. As such, the present disclosure should be construed to extend to any modifications, equivalents, and substitutes in addition to those which are particularly set out in the accompanying drawings and the corresponding description. Usage of words such as first, second, third etc., to describe components/elements/steps is for the purposes of this description and should not be construed as sequential ordering/placement/occurrence unless specified otherwise.

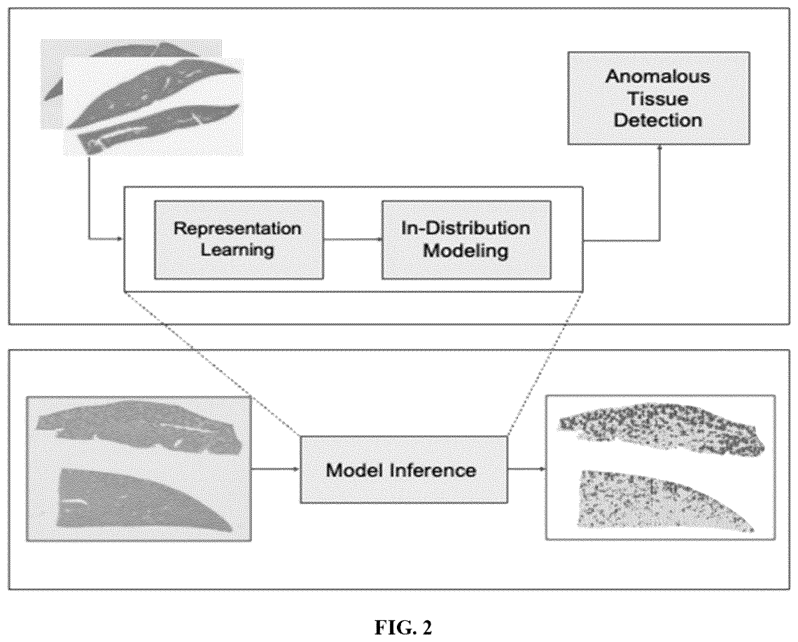

The embodiments herein achieve methods and systems for performing unsupervised out-of-distribution detection of abnormal regions in histopathology media using multi-class in-distribution modelling. Referring now to the drawings, and more particularly to FIGS. 2 through 6B, where similar reference characters denote corresponding features consistently throughout the figures, there are shown embodiments.

Embodiments herein disclose methods and system for performing unsupervised detection of one or more anomalies in histopathology media. Within the context of drug development, embodiments herein pertain to nonclinical toxicological pathology studies. In the clinical context, embodiments herein specifically refer to the identification of tumours in cancer pathology. Embodiments herein focus on automatically identifying abnormal regions in a tissue whole slide media by utilizing a multi-class normal representation that is learned exclusively from normal tissue whole slide media.

The embodiments described herein propose a multi-class in-distribution model to address the limitations of traditional anomaly detection methods by learning the distribution of different normal tissue classes. Instead of treating all normal tissue as a single, unified category, this approach trains a deep learning model to recognize and differentiate multiple normal tissue subtypes, ensuring a more comprehensive representation of normal histological variations.

Media as referred to herein can be one of an image, a video, a slideshow, an animation, and so on.

FIG. 2 depicts an example process for performing unsupervised out-of-distribution detection of abnormal regions in histopathology media using multi-class in-distribution modelling. A digital whole slide media of the stained tissue is acquired by utilizing digital whole slide scanners. In one scenario, embodiments herein comprise techniques and systems for automatically evaluating toxicologic histopathology whole slide media to detect tissue abnormalities and evaluate drug-induced toxicity in laboratory animals. Embodiments herein detect one or more malignant areas in a needle core biopsy or surgical tissue sample within the cancer care procedure. Embodiments herein involve acquiring normal Whole Slide Images (WSIs) or WSIs that have substantial normal regions.

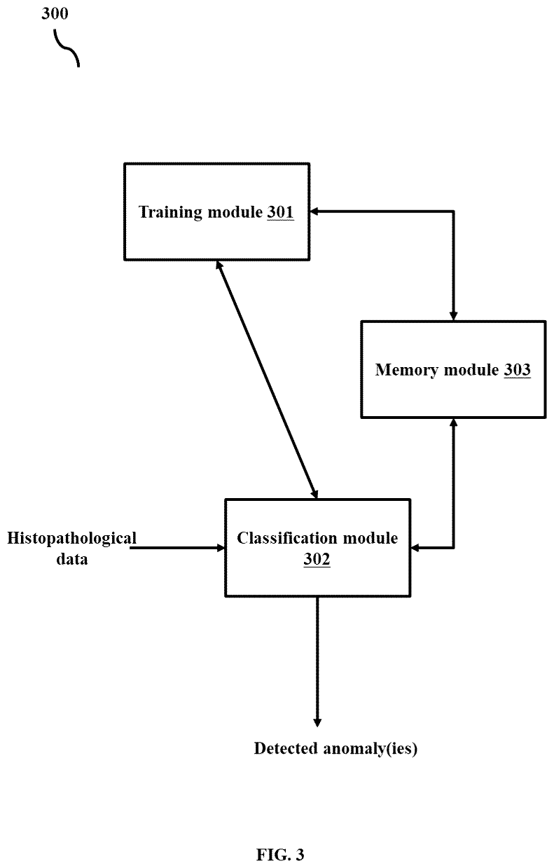

FIG. 3 depicts a system for performing unsupervised out-of-distribution detection of abnormal regions in a tissue sample. The system 300, as depicted, comprises a training module 301, a classification module 302, and a memory module 303.

In the embodiment shown herein, the memory module 303may comprise one or more volatile and non-volatile memory components that are capable of storing data and instructions to be executed. Examples of the memory module 303 can be, but are not limited to, NAND, embedded Multimedia Card (eMMC), Secure Digital (SD) cards, Universal Serial Bus (USB), Serial Advanced Technology Attachment (SATA), solid-state drive (SSD), and so on. The memory module 303 may also include one or more computer-readable storage media. Examples of non-volatile storage elements may include magnetic hard discs, optical discs, floppy discs, flash memories, or forms of electrically programmable memories (EPROM) or electrically crasable and programmable (EEPROM) memories. In addition, the memory module 303 may, in some examples, be considered a non-transitory storage medium. The term “non-transitory” may indicate that the storage medium is not embodied in a carrier wave or a propagated signal. However, the term “non-transitory” should not be interpreted to mean that the memory module 303 is non-movable. In certain examples, a non-transitory storage medium may store data that can, over time, change (for example, in Random Access Memory (RAM) or cache). In an embodiment herein, the memory module 303 can present in at least one of the training module 301 and/or the classification engine 302. In an embodiment herein, the memory module 303 can be at least one of the cloud, a data server, a file server, a Network-attached storage (NAS), a network storage, or any other suitable data storage location.

The training module 301 can be at least one of a single processor, a plurality of processors, multiple homogeneous or heterogeneous cores, multiple Central Processing Units (CPUs) of different kinds, microcontrollers, special media, and other accelerators. The training module 301 may be an Application Processor (AP), a graphics-only processing unit such as a Graphics Processing Unit (GPU), a Visual Processing Unit (VPU), and/or an Artificial

Intelligence (AI)-dedicated processor such as a Neural Processing Unit (NPU). In an embodiment herein, the training module 301 can be a dedicated control unit. In an embodiment herein, the training module 301 can be a control unit, which performs one or more actions/tasks in addition to embodiments as disclosed herein.

Training the classification model can comprise of the training module 301 learning one or more feature representations for different normal classes of the tissue, capturing the distinct structural and morphological characteristics of each subtype of histopathology related tissue. In an example scenario, if the tissue is from the kidney tissue, then examples of the classes, as referred to herein, can be, but not limited to, cortex, medulla, papilla, and so on. Using the learned representations, the training module 301 can define an in-distribution space, wherein the in-distribution space corresponds to the normal tissue distribution. The in-distribution space can serve as a reference for distinguishing normal from abnormal tissue.

Using contrastive learning, the training module 301 can constrain the in-distribution space. Contrastive learning can be used by the training module 301 to enhance the model's ability to distinguish between different normal tissue types, while ensuring that samples of similar tissue are clustered together. This contrastive distribution learning can help improve the discriminability of normal representations, reducing overlap between different normal classes of the tissue, and enable the classification module 302 to perform anomaly detection more robustly. The training module 302 can store the trained classification model in a suitable location, such as, but not limited to, the memory module 303.

The classification module 302 can be at least one of a single processor, a plurality of processors, multiple homogeneous or heterogeneous cores, multiple Central Processing Units (CPUs) of different kinds, microcontrollers, special media, and other accelerators. The classification module 302 may be an Application Processor (AP), a graphics-only processing unit such as a Graphics Processing Unit (GPU), a Visual Processing Unit (VPU), and/or an Artificial Intelligence (AI)-dedicated processor such as a Neural Processing Unit (NPU). In an embodiment herein, the classification module 302 can be a dedicated control unit. In an embodiment herein, the classification module 302 can be a control unit, which performs one or more actions/tasks in addition to embodiments as disclosed herein.

The classification module 302 can access the histopathology media of a tissue sample from at least one of the memory module 303, from a scanning device (not shown) directly in real time (wherein the scanning device is capturing the histopathology media in real time).

The classification module 302 can use the classification model to analyze the tissue sample. Initially, the classification module 302 can extract one or more feature representations from the tissue samples, as depicted in the histopathology media. Using the classification model, the classification module 302 can determine whether a given sample falls within the normal distribution or deviates as an anomaly. In an embodiment herein, the classification module 302 can determine whether a given sample falls within the normal distribution or deviates as an anomaly using a distance measured between one or more features from one or more test whole slide images and each of the normal class distributions (as determined from the classification model). In an embodiment herein, the classification module 302 can determine a Euclidean distance between one or more test WSI features and each of the normal class distributions. In an embodiment herein, the classification module 302 can determine a Mahalanobis distance between one or more test WSI features and each of the normal class distributions. The classification module 302 can check if the determined distance has a deviation from all normal class distributions greater than a pre-defined threshold. In an embodiment herein, the pre-defined threshold can be defined based on statistical distribution of the normal class data. If the determined distance has a deviation from all normal class distributions greater than a pre-defined threshold, the classification module 302 can determine that there is at least one anomaly/abnormality in the tissue sample. This approach can be effective for detecting out-of-distribution (OOD) tissue patterns.

By leveraging multi-class in-distribution modelling and contrastive learning, embodiments herein significantly improve the precision of anomaly detection in histopathology, reducing false positives and ensuring better differentiation between normal and abnormal tissue structures.

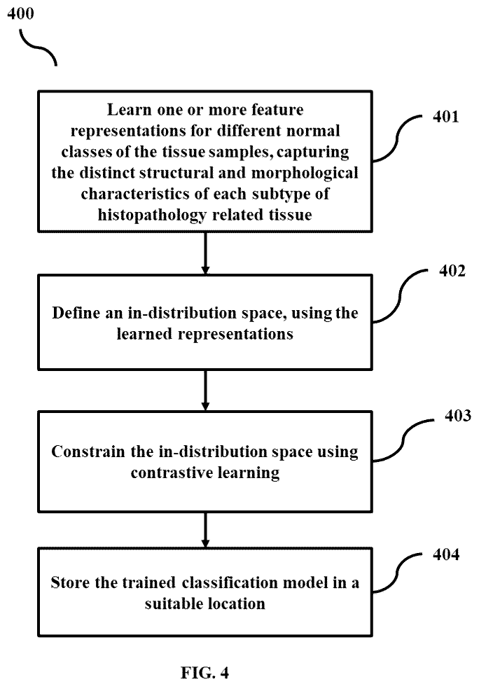

FIG. 4 is a flowchart depicting the process of training a classification model. In step 401, the training module 301 learns one or more feature representations for different normal classes of the tissue samples, capturing the distinct structural and morphological characteristics of each subtype of histopathology related tissue. Using the learned representations, in step 402, the training module 301 defines an in-distribution space, wherein the in-distribution space serves as a reference for distinguishing normal from abnormal tissue. Using contrastive learning, in step 403, the training module 301 constrains the in-distribution space. Contrastive learning enhances the model's ability to distinguish between different normal tissue types, while ensuring that samples of similar tissue are clustered together. This contrastive distribution learning helps improve the discriminability of normal representations, reducing overlap between different classes, and enables the classification module 302 to perform anomaly detection more robustly. In step 404, the training module 302 stores the trained classification model in a suitable location, such as, but not limited to, the memory module 303. The various actions in method 400 may be performed in the order presented, in a different order or simultaneously. Further, in some embodiments, some actions listed in FIG. 4 may be omitted.

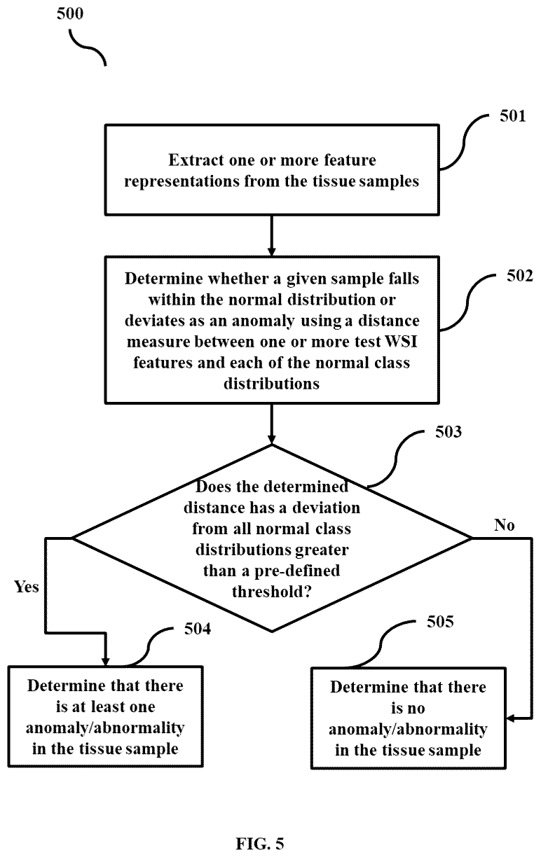

FIG. 5 is a flowchart depicting the process of detecting at least one anomaly/abnormality in a tissue sample. In step 501, the classification module 302 extracts one or more feature representations from the tissue samples, as depicted in the histopathology media. Using the classification model, in step 502, the classification module 302 determines whether a given sample falls within the normal distribution or deviates as an anomaly using a distance measured (which can be for example, a Euclidean distance, a Mahalanobis distance) between one or more test WSI features and each of the normal class distributions (as determined from the classification model). In step 503, the classification module 302 checks if the determined distance has a deviation from all normal class distributions greater than the pre-defined threshold. If the determined distance has a deviation from all normal class distributions greater than a pre-defined threshold, in step 504, the classification module 302 determines that there is at least one anomaly/abnormality in the tissue sample. If the determined distance has a deviation from all normal class distributions which is not greater than a pre-defined threshold, in step 505, the classification module 302 determines that there is no anomaly/abnormality in the tissue sample. This approach can be effective for detecting out-of-distribution (OOD) tissue patterns. The various actions in method 500 may be performed in the order presented, in a different order or simultaneously. Further, in some embodiments, some actions listed in FIG. 5 may be omitted.

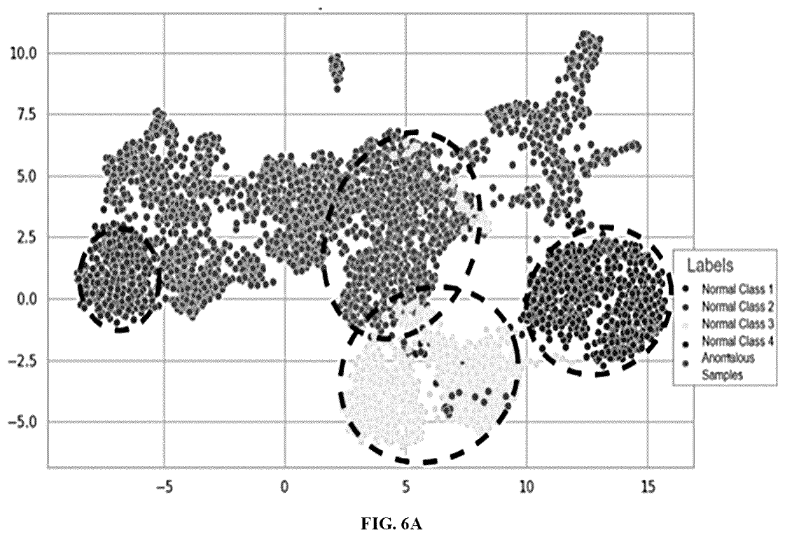

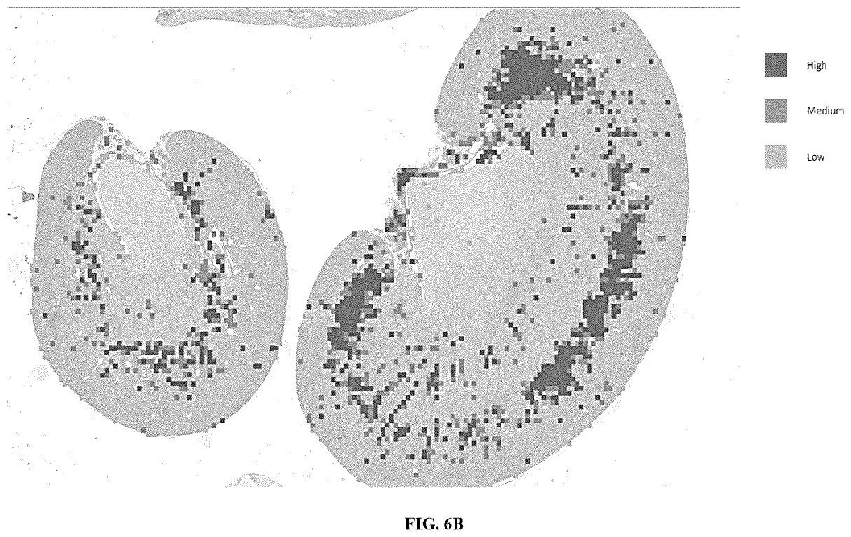

Embodiments herein involve generating a heatmap representation of abnormal regions based on the distance measure. Embodiments herein can cluster abnormal regions based on distance into low, medium and high probability abnormal regions. FIG. 6A presents a two dimensional (2D) representation of a multi-class normal distribution alongside abnormal class samples, visually distinguishing normal variations from anomalies. This distribution serves as a reference framework to understand deviations in biological tissue characteristics. FIG. 6B overlays clustered abnormal class samples specifically from kidney tissue, illustrating how the unsupervised model detects and groups abnormalities. This visualization highlights distinct clusters within the abnormal category, aiding in the differentiation of pathological findings from normal variations. The clustering provides insights into the nature and extent of anomalies within kidney tissue, showcasing the effectiveness of the model in identifying previously unlearned patterns.

The embodiments disclosed herein can be implemented through at least one software program running on at least one hardware device and performing network management functions to control the network elements. The elements include blocks which can be at least one of a hardware device, or a combination of hardware device and software module.

The embodiments disclosed herein describe methods and systems for performing unsupervised out-of-distribution detection of abnormal regions in histopathology media using multi-class in-distribution modelling. Therefore, it is understood that the scope of the protection is extended to such a program and in addition to a computer readable means having a message therein, such computer readable storage means contain program code means for implementation of one or more steps of the method, when the program runs on a server or mobile device or any suitable programmable device. The method is implemented in at least one embodiment through or together with a software program written in e.g., Very high speed integrated circuit Hardware Description Language (VHDL) another programming language, or implemented by one or more VHDL or several software modules being executed on at least one hardware device. The hardware device can be any kind of portable device that can be programmed. The device may also include means which could be e.g., hardware means like e.g., an ASIC, or a combination of hardware and software means, e.g., an ASIC and an FPGA, or at least one microprocessor and at least one memory with software modules located therein. The method embodiments described herein could be implemented partly in hardware and partly in software. Alternatively, the invention may be implemented on different hardware devices, e.g., using a plurality of CPUs.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of embodiments and examples, those skilled in the art will recognize that the embodiments and examples disclosed herein can be practiced with modification within the scope of the embodiments as described herein.

What is claimed is:

1. A method (500) for detecting at least one anomaly in a tissue sample, the method comprising:
extracting (501), by a classification module (302), one or more feature representations from the tissue sample;
determining (502), by the classification module (302), whether a given sample falls within a normal distribution or deviates as an anomaly using a distance measured between one or more test WSI features and each of normal class distributions using a classification model; and
determining (504), by the classification module (302), that there is at least one anomaly in the tissue sample, if the determined distance has a deviation from all normal class distributions greater than a pre-defined threshold.

2. The method, as claimed in claim 1, wherein the distance is one of a Euclidean distance, and a Mahalanobis distance.

3. The method, as claimed in claim 1, wherein the method comprises determining (505), by the classification module (302), that there is no anomaly in the tissue sample, if the determined distance has a deviation from all normal class distributions which is not greater than a pre-defined threshold.

4. The method, as claimed in claim 1, wherein a method (400) for training the classification model comprises:
learning (401), by a training module (301), one or more feature representations for different normal classes of a plurality of tissue samples, wherein the one or more feature representations capture one or more distinct structural and morphological characteristics of each subtype of histopathology related tissue;
defining (402), by the training module (302), an in-distribution space using the one or more learned feature representations; and
constraining (403), by the training module (302), the in-distribution space using contrastive learning.

5. A system (300) for detecting at least one anomaly in a tissue sample, the system (300) comprising:
a classification module (302), wherein the configuration module (302) is configured to:
extract one or more feature representations from the tissue sample;
determine whether a given sample falls within a normal distribution or deviates as an anomaly using a distance measured between one or more test WSI features and each of normal class distributions using a classification model; and
determine that there is at least one anomaly in the tissue sample, if the determined distance has a deviation from all normal class distributions greater than a pre-defined threshold.

6. The system, as claimed in claim 5, wherein the distance is one of a Euclidean distance, and a Mahalanobis distance.

7. The system, as claimed in claim 5, wherein the classification module (302) is configured to determine that there is no anomaly in the tissue sample, if the determined distance has a deviation from all normal class distributions which is not greater than a pre-defined threshold.

8. The system, as claimed in claim 5, wherein the system (300) further comprises a training module (301), wherein the training module (301) is configured to:
learn one or more feature representations for different normal classes of a plurality of tissue samples, wherein the one or more feature representations capture one or more distinct structural and morphological characteristics of each subtype of histopathology related tissue;
define an in-distribution space using the one or more learned feature representations; and
constrain the in-distribution space using contrastive learning.
